(19) United States
(12) Patent Application Publication
(10) Pub. No.: US 2006/0067912 A1
(43) Pub. Date: Mar. 30, 2006

(54) Methods for the induction of professional and cytokine-producing regulatory T cells

(75) Inventor: David A. Horwitz, Santa Monica, CA (US)

Correspondence Address: Richard F. Trecartin;Dorsey & Whitney LLP, Intellectual Property Department, 555 California Street, Suite 1000, San Francisco, CA, 94104-1513, US

(73) Assignee: University of Southern California

(21) Appl. No.: 11/241,467

(22) Filed: Sep. 30, 2005

Related U.S. Application Data
(63) Continuation of application No. 10/326,850, filed on Dec. 20, 2002, now abandoned.
(63) Continuation-in-part of application No. 10/028,944, filed on Dec. 21, 2001, now Pat. No. 6,797,267.
Application No. 10/028,944 is a continuation of application No. 09/564,436, filed on May 4, 2000, now Pat. No. 6,358,506.
(60) Provisional application No. 60/342,655, filed on Dec. 21, 2001.
(60) Provisional application No. 60/374,102, filed on Apr. 19, 2002.
(60) Provisional application No. 60/132,616, filed on May 5, 1999.

(51) Int. Cl. A61K 39/395; A61K 38/20; A61K 35/14

(52) U.S. Cl. 424/85.2; 424/93.7; 424/144.1

(57) ABSTRACT

The field of the invention is generally related to methods used for the induction of T cells with suppressive activity. More specifically, the methods are used to generate professional regulatory T cells and cytokine-producing T cells with enhanced suppressive activity.

This application claims the benefit of the filing date of U.S. Ser. No. 60/342,655, filed Dec. 21, 2001 and U.S. Ser. No. 60/374,102, filed Apr. 19, 2002, and is a continuation in part of 10/028,944, filed Dec. 21, 2001 now U.S. Pat. No. 6,797,267, which is a continuation of Ser. No. 09/564,436, filed May 4, 2000 now U.S. Pat. No. 6,358,506, which claims the benefit of the filing date of U.S. Ser. No. 60/132,616, filed May 5, 1999.

### FIELD OF THE INVENTION

The field of the invention is generally related to methods used for the induction of T cells with suppressive activity. More specifically, the methods are used to generate professional regulatory T cells and cytokine-producing T cells with enhanced suppressive activity.

### BACKGROUND

A number of immune disorders can be characterized by the failure of the immune system to distinguish self from non-self. For example, autoimmune diseases are caused by the failure of the immune system to distinguish self from non-self. In these diseases, the immune system reacts against self tissues and this response ultimately causes inflammation and tissue injury. Autoimmune diseases can be classified into two basic categories: antibody-mediated diseases such as systemic lupus erythematosus (SLE), pemphigus vulgaris, myasthenia gravis, hemolytic anemia, thrombocytopenia purpura, Grave's disease, Sjogren's disease and dermatomyositis; and cell-mediated diseases such as Hashimoto's disease, polymyositis, disease inflammatory bowel disease, multiple sclerosis, diabetes mellitus, rheumatoid arthritis, and scleroderma.

Alternatively, the ability of the immune system to recognize and respond to foreign antigens is undesirable in some situations. For example, the rejection of solid organ transplants, i.e., graft rejection, occurs when the immune system of the recipient recognizes foreign histocompatibility antigens. Likewise, transplantation of hematopoietic stem cells from an unrelated (or allogeneic) donor can trigger a deadly response called graft versus host disease (GVHD) because the donor stem cell preparations generally contain T lymphocytes. GVHD results when the donor T lymphocytes recognize histocompatibility antigens of the recipient as foreign and respond by causing multi-organ dysfunction and destruction.

Methods for alleviating the symptoms of autoimmune disorders and for preventing graft rejection typically involve the use of steroids with potent anti-inflammatory and immunosuppressive action, such as prednisone. Other strong immunosuppressive drugs that can be used include azathioprine, cyclosporin, and cyclophosphamide. All of these drugs have undesirable side effects due to the global reduction of the immune system.

A more desirable strategy would be to identify methods that do not have undesirable side effects. Methods for “resetting” the immune system, by generating regulatory T cells (also referred to as suppressor T cells) are described in U.S. Pat. Nos. 6,228,359, and 6,358,506, 6,557,765 and U.S. Ser. Nos. 09/653,924 and 09/833,526, all of which are incorporated herein by reference in their entirety. These methods are directed towards restoring normal regulatory cell function in an affected individual.

Accordingly, it is an object of the present invention to provide methods for treating peripheral lymphoid tissue for the generation of regulatory T cells that can be used to treat autoimmune disorders, as well prevent immune responses resulting in graft rejection and graft versus host disease.

### SUMMARY OF THE INVENTION

In accordance with the objects outlined above, the present invention provides compositions and methods that can be used to generate regulatory T cells in a sample of ex vivo peripheral blood mononuclear cells (PBMCs). The regulatory cells so generated may be professional CD4+ CD25+ regulatory cells or cytokine-producing regulatory cells. Preferably, both the number and suppressive activity of the regulatory cells are increased.

The regulatory compositions may comprise a number of components, including cytokines, T cell activators, T cell stimulators, non T accessory cells, and neutralizing anti-cytokine antibodies. These components may be added in any number of combinations including one or more compounds from the same class of compounds, i.e., two or more cytokines, may be mixed together. The composition also may contain compounds from different classes of compounds, such as a one or more cytokine, T cell activator, etc.

In an additional aspect, the present inventions provides methods for inhibiting aberrant or undesirable immune responses comprising administering the regulatory T cells generated using the regulatory compositions described herein.

In a further aspect, the present invention provides kits for the practice of the methods of the invention, i.e., the incubation of cells with the regulatory compositions.

### BRIEF DESCRIPTION OF THE FIGURES

FIGS. 1A-C depict some preferred embodiments for generating regulatory T cells beginning with a population of T cells (1) comprising mostly conventional T cells (2) and virgin professional regulatory T cells (3). Targeting virgin CD4+ CD25+ cells with a regulatory composition results in a treated T cell population (6) comprising >50% activated professional regulatory CD4+ CD25+ T cells with enhanced suppressive activity (4). These cells may be the progeny of the virgin regulatory cells or other T cells that have been induced to develop potent contact-dependent suppressive activity (FIG. 1A).

FIG. 1B depicts another preferred embodiment comprising mixing activated professional regulatory T cells (4) with conventional T cells (2) and stimulating with a T cell activator. In this embodiment, the professional regulatory cells induce other T cells to become cytokine-producing T cells (5) by a phenomenon called T cell tolerance.

In another embodiment, treatment of conventional T cells (2) depleted of CD4+ CD25+ with a regulatory composition induces these cells to become activated cytokine producing regulatory T cells (5) with enhanced suppressive activity (FIG. 1C).

FIGS. 2A and B illustrate that TGF-β co-stimulation markedly increases the percentage and absolute numbers of total CD4+ CD25+ and CD4+ CD25− cells. Does dependent effects of TGF-β are shown. The cytokines produced by T cells co-stimulated by TGF- also increase CD8+ cells.

FIG. 3 depicts that the suppressive effects of TGF-β may be overcome by neutralizing IL-2 with a monoclonal antibody. The dose-dependent co-stimulatory properties of TGF-β were abolished with small amounts of anti-IL-2. These doses did not affect T cell activation without TGF-β. Larger amounts of anti-IL-2 completely inhibited IL-2 activity, which in turn led to suppressive effects on TGF-β.

FIGS. 4A-F show that the combination of IL-2 and TGF-β stimulates professional CD4+ CD25+ cells and increases their suppressive activity. Using alloantigens as the T cell activator, this is demonstrated for the small “virgin” CD25+ subset isolated from the naïve fraction of CD4+ CD45RA+ cells (FIGS. 4A and B). Suppression was assessed by the inhibiting the generation of cytotoxic T lymphocyte (CTL) activity against the lymphoblasts of the stimulator using the well established chromium release assay with three effector to target cell ratios. Most CD4+ CD25+ cells are contained in the previously activated, or memory CD45RO+ subset. Studies showing the suppressive activity of these regulatory cells are shown in FIGS. 4C and 4D. The suppressive effects of memory CD4+ CD25+ cells alloactivated for 5 days with IL-2 (10 U/ml)±TGF-beta 1 (1 ng/ml) on other T cells is shown. The mixed lymphocyte was repeated with the same donors and the activated CD4+ CD25+ cells were added to autologous responder T cells in a 1:10 ratio. The responder cells were labeled with carboxyfluorescein and the percentage and absolute numbers of cycling CD8+ cells was assessed 6 days after activation. The clear bar indicates cycling CD8+ cells without added activated CD4+ CD25+ cells. The gray bar indicates the effect of CD4+ CD25+cells activated with IL-2, and the black bar the effect of cells activated with IL-2 and TGF-β. The suppressive activity is not decreased by anti-TGF-β. Using inhibition of the generation of allo-CTL activity to assess suppressive activity, the combination of IL-2 and TGF-β increases the cytokine-dependent suppressive activity of conventional naïve CD4+ CD45RA+CD25− cells and of previously activated or memory CD4+ CD45RO+ CD25− cells (FIGS. 4E and F). Anti-TGF-β abolishes the suppressive effects of the cells by increasing CTL activity to levels observed with control CD4+ cells. The suppressive activity induced by the superantigen, staphylococcal enterotoxin B, in the presence of TGF-β is also abolished by anti-TGF-β. In this example, conventional, CD4+ CD25− cells are induced to become regulatory cells.

FIGS. 5A-D confirm the critical role of the CD25+ subset in mediating the co-stimulatory effects of TGF-β. Naïve CD4+ CD45RO− cells were prepared and these were further fractionated into CD25+ and CD25− cells by cell sorting. These cells were activated in the allogeneic mixed lymphocyte reaction with IL-2 (10 U/ml)±TGF-beta 1 (1 ng/ml). FIG. 5A shows that the TGF-β mediated 2 fold increase in cell number was abolished by removal of the <1% CD25+ cells. The addition of CD25+ cells to CD25− cells in a 1:10 ratio restored this effect. FIGS. 5B and C show a similar experiment where TGF-β induced enhancement of cell numbers was only modest, but the phenotype of the cells was markedly altered. TGF-β markedly increased the number of cells expressing both CD25 and CTLA-4. This effect was lost when the CD25+ subset was removed. The addition of CD25+ cells to CD25− cells in a ratio of 1:10 restored the effect. FIG. 5D shows that when the cells were restimulated without TGF-β, those preparations containing CD25+ cells markedly increased in comparison with those where the CD25+ subset had been removed.

FIG. 6 shows an experiment suggesting that TGF-β conditioned, activated CD4+ CD25+ cells can induce conventional CD4+ CD25− cells to develop suppressive activity. The experimental design is similar to that described in FIG. 5. Naïve CD4+ cells, purified CD25+ cells, CD25 depleted cells and a mixture of CD25+ and CD25− cells that had been alloactivated with and without TGF-β were added to fresh T cells in a 1:10 ratio and alloactivated with the same allogeneic stimulator cells. The percentage of CD8+ CD25+ cells after 6 days of culture was determined. The horizontal line shows the percentage of CD8+ CD25+ cells without added CD4+ cells. Marked suppression by CD25+ cells, but not by CD25− cells is shown. When CD25+ cells were mixed with CD25− cells in a ratio of 1:10 and alloactivated with TGF-β, their suppressive activity was equivalent to a similar number of purified CD4+ CD25+ cells. This experiment also illustrates that the suppressive-inducing property of IL-2 and TGF-β is not exclusively limited to the CD25+ subset of CD4+ cells. IL-2 and TGF-β treatment of naïve CD4+ CD45RA+ cells that had been depleted of CD25+ cells also induced suppressive activity, The suppression was not abolished with anti-TGF-β.

FIGS. 7A and B illustrate another experiment where IL-2 and TGF-β induce CD4+ CD45RA+ CD25− cells to become suppressor cells. Various CD4+ CD45RA+ subsets were activated ±TGF-β and tested for suppressive activity by inhibition of the generation of CTL activity. In this experiment, the suppressive activity of total CD4+ CD45RA+ cells was identical to the CD4+ CD45RA+ CD25− subset (FIG. 7A). Anti-TGF-β did not reverse this suppressive activity (FIG. 7B).

FIGS. 8A-J demonstrate that IL-2 and TGF-β enhance the growth and suppressive activity of the previously activated or memory fraction of CD4+ CD45RO+ CD25+ cells after the endogenous suppressive effects are overcome. The endogenous negative feedback effects of CD4+ CD45RO+ CD25+ cells on suppressor cell induction was shown by alloactivating total CD4+ cells with TGF-β to generate regulatory cells. Inhibition of the generation of CTL activity was used to assess suppression. TGF-β show that the suppressive activity developed by total CD4+ cells was significantly less than when the CD45RO subset, or the CD25+ fraction was depleted (FIGS. 8A and B). FIG. 8C shows that the removal of the CD25+ suppressor cells correspondingly increased the number of CD4+ cells following alloactivation. The horizontal line indicates the starting number of CD4+ CD45RO+ cells. Because most CD4+ CD25+ cells are contained in the CD45RO fraction, the TGF-β consistently decreased the number of T cells recovered following the mixed lymphocyte reaction. FIGS. 8D-F show that IL-2 can overcome the inhibitory effects of TGF-β. CD4+ CD45RO cells were prepared and some were depleted of CD25+ cells. The two populations were stimulated with allogeneic cells with IL-2 and TGF-β as described above. The total number and those express CD25 and CTLA-4 after 6 days was measured. In comparison with IL-2 alone, the combination of IL-2 and TGF-β modestly, but significantly enhanced the total number (FIG. 8D), the number of CD25+ cells (FIG. 8E), and the number of CTLA-4+ cells (FIG. 8F). By contrast, TGF-β had inhibitory effects on CD4+ CD45RO+ CD25− cells. These studies suggested that the co-stimulatory effects of IL-2 and TGF-specifically targeted the CD4+ CD45RO+ CD25 subset. FIG. 8G illustrates that the combination of IL-2 and TGF-β enhances the growth of CD4+ CD45RO+ CD25+ cells. The horizontal line indicates the starting population of cells. While the numbers of these CD25+ T cells decreased by 50% in medium, with IL-2, and with TGF-β, they increased by 33% when cultured with IL-2 and TGF-β. FIG. 8H shows TGF-β-dependent up regulation of CD122. CD25+ were targeted and depletion of CD25+ abolished this effect. FIGS. 8I and J show that even CD25+ depleted naïve CD4+ could be induced to develop suppressive activity independent of TGF-β. The addition of IL-2 to TGF-β enabled a CD4+ CD45RA+ CD25− cells to develop potent suppressive activity that could not be abolished by anti-TGF-β.

FIGS. 9A-D illustrate the co-stimulatory effects of TGF-β on the expression of surface markers which are characteristically displayed by professional CD4+ CD25+ regulatory T cells. Besides CD25, these include CTLA-4 and CD62L. The total numbers of CD25+ cells, CTLA-4+ cells and CD62L cells were increased in cultures containing TGF-β (FIGS. 9A, B, C). In addition, TGF-β enhanced expression of CD122, the beta chain of the IL-2 receptor (FIG. 9D). This receptor binds IL-2 and IL-15 and is involved in signal transduction. The CD4+ CD25+ subset was the specific target of TGF-β as depletion of this subset abolished the marked enhancement of receptor expression.

FIG. 10 illustrates that blocking the beta chain of the IL-2 receptor with anti-122 may abolish the suppressive activity of CD25+ cells. T cells were stimulated with allogeneic cells and CTL activity was assessed. Partial depletion of CD25+ cells with immunomagnetic beads enhanced CTL activity, suggesting that total suppressor cell activity was diminished. Blocking IL-2 signaling through the IL-2Rbeta chain with various doses of anti-CD122 enhanced CTL activity even greater. This effect was due to blocking the activity of T cells with lower amounts of cell-surface CD25, and inhibiting suppressor cells that had lost expression of CD25.

FIGS. 11A and B illustrate that in addition to the combination of IL-2 and TGF-β, IL-15 and TGF-β has co-stimulatory effects on CD4+ cells. CD4+ cells were stimulated with allogeneic non-T cells±TGF-β and expression of CD122 and CD25 CTLA-4 double positive cells were quantified by flow cytometry. Adding IL-15 to TGF-β enhanced expression of these markers as well as the combination of IL-2 and TGF-β.

### DETAILED DESCRIPTION

The present invention is directed to methods of generating regulatory T cells ex vivo. The methods involve isolating naïve T cells and treating them with a regulatory composition. Treatment with a regulatory composition increases the numbers and suppressive activity of the generated regulatory T cells. This enhanced suppressive activity is thought to be mediated through the induction of a cell surface receptor critical for T cell proliferation and differentiation.

Moreover, the regulatory T cells generated by the methods described herein induce other T cell populations to develop regulatory cells with enhanced suppressive activity. This ability to induce suppressive activity may occur through a phenomenon called infectious tolerance (see Waldmann H., et al, (1993) Science, 259: 974-7). Regulatory T cells prevent self-reactive T cells from becoming activated and causing immune pathology. Regulatory T cells also prevent microbial antigens from inducing immune-mediated tissue injury (see Zheng, et al., (2002) J. Immunol., 169: 4183-4189).

Several regulatory T cell subsets have been identified (see Zheng, et al., (2002) J. Immunol., 169: 4183-4189). For example, in the thymus, a subset of CD4+ T cells (CD4+ CD25+) constitutively express the alpha chain of the IL-2 receptor complex (CD25). These CD4+ cells have a broad range of suppressive activities that include prevention of autoimmunity and graft rejection, control of homeostatic lymphocyte proliferation, and regulation of germinal center formation in lymph nodes. These cells suppress by a contact-dependent, TGF-β independent, mechanism and are referred to as “professional” regulatory T cells.

Other regulatory cells, called Th3 or Th3-like cells, can be either CD4+ or CD8+ T cells that produce immunosuppressive levels of TGF-β. These cells are produced in peripheral lymphoid tissue, such as mucosal lymphoid tissues in response to oral or intranasal immunization. Th3 cells have a protective role in several experimental autoimmune diseases, including experimental allergic encephalitis and diabetes mellitus.

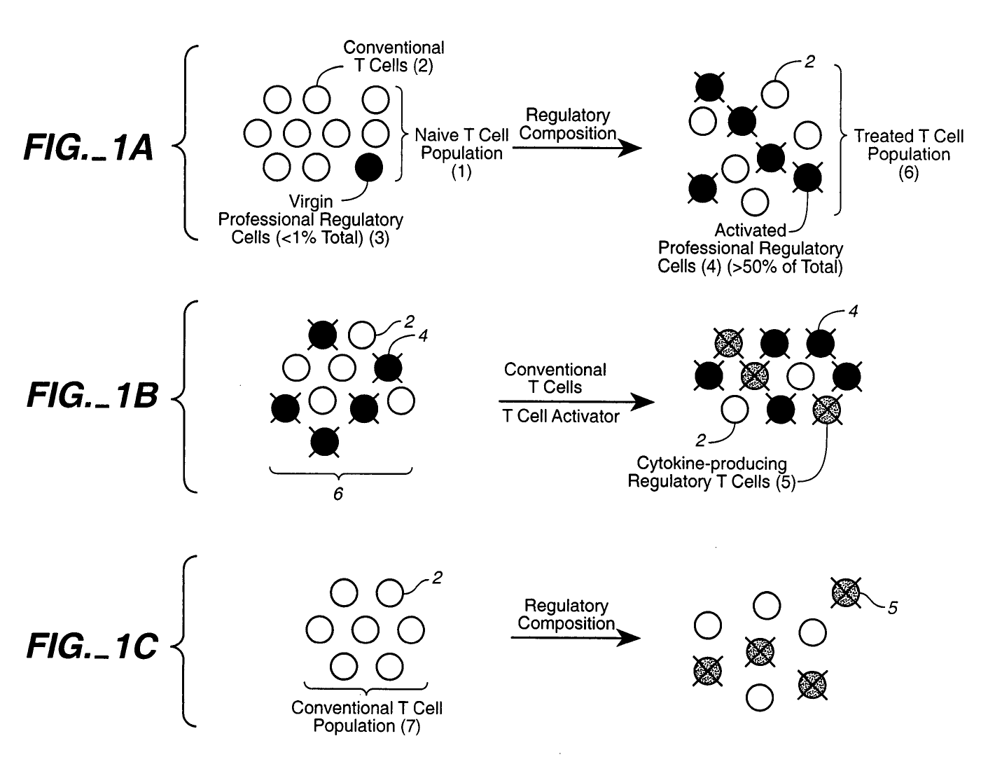

When a naïve CD4+ cell population comprising primarily conventional T cells (CD4+ CD25−) and a few virgin CD4+ CD25+ professional regulatory T cells is treated with a regulatory composition, some of the virgin professional regulatory T cells are stimulated to proliferate, resulting in the generation of activated professional regulatory cells with enhanced suppressive activity (see FIG. 1A). As illustrated in FIG. 1A, naïve (CD45RA+RO−) CD4+ cells are generally 99% conventional T cells (CD4+ CD25−). However, when activated with a regulatory composition, the percentage of CD4+ CD25+ cells is markedly increased, such that these cells now comprise greater than 50% of the total treated CD4+ cell population. The CD4+ CD25+ population comprises the progeny of the virgin CD4+ CD25+ cells and other T cells that were activated by the virgin CD4+ CD25+ cells to become regulatory cells. This phenomenon is dependent upon the virgin CD4+ CD25+ regulatory cell subset as it can be abrogated by removing this subset (Yamagiwa et al, (2001) J. Immuno. 166: 7282-89).

When activated professional regulatory T cells are mixed with conventional T cells, they generate a population of cytokine producing regulatory T cells when treated with a T cell activation (FIG. 1B). Significantly, repeated treatment with a regulatory composition comprising cytokines is not needed to generate this population of cytokine producing regulatory T cells.

Cytokine-producing regulatory T cells may also be generated from conventional T cells depleted of professional CD25+ T cells by treating the conventional T cells with a regulatory composition (see FIG. 1C).

Thus, by targeting a small number of virgin CD4+ CD25+ cells in T cell preparations with a regulatory composition results in (1) a marked expansion of professional regulatory T cells with enhanced suppressive activity; and (2) induction of conventional T cells to become cytokine producing cells. The proportions of professional and cytokine-producing cells that are generated depends upon the composition of the T cell population that is treated, the composition of the regulatory composition and whether non T accessory cells are added.

Administration of one or more of the regulatory T cell subsets generated by one of above approaches can be used to inhibit undesirable immune responses in an individual. For example, in individuals with antibody-mediated autoimmune disorders, the present invention restores the capacity of peripheral blood T cells to down regulate antibody production and restores cell mediated immune responses. In patients with cell-mediated disorders, the present invention generates regulatory T cells that suppress cytotoxic T cell activity in other T cells. In patients receiving a solid organ transplant, the present invention prevents the recipient's T cells from destroying the donor organ. In patients receiving a stem cell transplant, the present invention prevents the donor stem cells from destroying the recipient's cells and tissues.

Accordingly, the present invention is drawn to methods of generating regulatory T cells that comprise isolating T cells from peripheral blood mononuclear cells (PBMC) and treating those cells with a regulatory composition comprising at least one compound that induces the generation of regulatory T cells with suppressive activity.

Peripheral T Lymphocytes

In mature human immune systems, peripheral T cells, both CD4+ and CD8+, can be separated into two major subsets, naïve versus memory-effector, by a number of correlated functional and phenotypic features, including: (a) activation requirements; (b) effector function (e.g., cytokine synthesis); (c) homing behavior; (d) adhesion function; and, (e) cell surface phenotype. The putative naïve subset, which predominates in immature (i.e., neonatal) immune systems and resembles the most mature thymocytes demonstrates the following features: (a) little or no response to recall antigens; (b) little or no ability to produce effector cylines such as inferferon-γ; (c) high costimulatory requirements for TCR-mediated activation; (D) inefficient maturation into MHC-restricted cytotoxic T cells; (e) efficient in vivo localization in secondary lymphoid tissues but not tertiary sites; (f) relatively low susceptibility to apoptosis and, corresponding to these functional characteristics, (g) a predominance of the high molecular weight, low activity RA isoforms of the CD45 protein tyrosine phosphatase; (h) uniform low expression of many general adhesion molecules, such as CD11a/CD18(LFA-1), CD54 (ICAM-1), CD2, CD58 (LFA-3), and CD44, the apoptosis-triggering molecule CD95/FAX, and the tertiary skin-selective homing receptor CLA; (i) uniform high expression of the peripheral lymph node homing receptor L-selectin and the costimulatory molecule CD27; and (j) uniform moderate expression of the Peyer's patch homing receptor α4β7 integrin (Picker and Siegelman, (1999) “Lymphoid Tissues and Organs” in W. E. Paul, ed., Fundamental Immunology, 4^{th} ed., chapter 14, pp 479-531).

In contrast, the memory-effector subset, which contains the vast majority of cells capable of responding to recall antigens and can be generated in vitro from the aforementioned naïve subset following appropriate activation, predominantly expresses the low molecular weight, high activity, RO CD45 isoform and shows efficient effector function (e.g., production of effector cytokines, cytotoxicity), increased CD95/FAS expression, and increased susceptibility to apoptosis, high levels of CD11a/CD18 and the other general adhesion molecules listed above, and heterogenous expression of the L-selectin, α4β7 integrin, and CD27 (Picker and Siegelman, (1999) “Lymphoid Tissues and Organs” in W. E. Paul, ed., Fundamental Immunology, 4^{th} ed., chapter 14, pp 479-531). Although the specific markers of memory-effector T-cell differentiation apply largely to humans, analogous naïve-memory T-cell dichotomy exists in animals as well.

Thus, the methods of the present invention begin with the isolation of T cells. As described below, the methods provide for the isolation of naïve and memory-effector T cells. By “naïve T cells” herein is meant T cells that express the CD45RA⁺/RO⁻, are undifferentiated because they have not been exposed to an immunizing antigen and have the characteristic features of “naïve T cells” discussed herein. By “memory-effector T cell” herein is meant T cells that express CD45RA⁻/RO⁺, are differentiated because they have been exposed to an immunizing antigen and have the characteristic features described above for “memory-effector” T cells.

Isolation of T Cells

Peripheral blood mononuclear cells (PBMC) are taken from heparinized venous blood of an individual using standard techniques (see Zheng, et al., (2002) J. Immunol., 169: 4183-4189). By “peripheral blood mononuclear cells” or “PBMC” herein is meant lymphocytes (including T-cells, B-cells, NK cells, etc.) and monocytes. Preferably, only PBMC are taken, either leaving or returning red blood cells to the patient. This is done as is known in the art, for example using leukophoresis techniques. In general, a 5 to 7 liter leukopheresis step is done, which essentially removes PBMC from a patient, returning the remaining blood components. Collection of the cell sample is preferably done in the presence of an anticoagulant such as heparin, as is known in the art.

In general, the sample comprising the PBMC can be pretreated in a wide variety of ways. Generally, once collected, the cells can be additionally concentrated, if this was not done simultaneously with collection or to further purify and/or concentrate the cells. The cells may be washed, counted, and resuspended in buffer.

The PBMCs are generally concentrated for treatment, using standard techniques in the art. In a preferred embodiment, the leukopheresis collection step results a concentrated sample of PBMCs, in a sterile leukopak, that may contain reagents or doses of the regulatory composition, as is more fully outlined below. Generally, an additional concentration/purification step is done, such as Ficoll-Hypaque density gradient centrifugation as is known in the art.

Separation or concentration procedures include but are not limited to magnetic separation, using antibody-coated magnetic beads, affinity chromatography, cytotoxic agents, either joined to a monoclonal antibody or used with complement, “panning”, which uses a monoclonal antibody attached to a solid matrix. Antibodies attached to solid matrices, such as magnetic beads, agarose beads, polystyrene beads, follow fiber membranes and plastic surfaces, allow for direct separation. Cells bound by antibody can be removed or concentration by physically separating the solid support from the cell suspension. The exact conditions and procedure depend on factors specific to the system employed. The selection of appropriate conditions is well within the skill in the art.

Antibodies may be conjugated to biotin, which then can be removed with avidin or streptavidin bound to a support, or fluorochromes, which can be used with a fluorescence activated cell sorter (FACS), to enable cell separation. Any technique may be employed as long as it is not detrimental to the viability of the desired cells.

In a preferred embodiment, the PBMC are separated in a automated, closed system. One such example is the Nexell Isolex 300i Magnetic Cell Selection System. Generally, a closed system is preferable to maintain sterility and to insure standardization of the methodology used for cell separation, activation and development of suppressor cell function.

In a preferred embodiment, the PBMC are washed to remove serum proteins and soluble blood components, such as autoantibodies, inhibitors, etc., using techniques well known in the art. Generally, this involves addition of physiological media or buffer, followed by centrifugation. This may be repeated as necessary. The PBMC can be resuspended in physiological media, preferably AIM-V serum free medium (Life Technologies) (since serum contains significant amounts of inhibitors of TGF-β) although buffers such as Hanks balanced salt solution (HBBS) or physiological buffered saline (PBS) can also be used.

In a preferred embodiment, peripheral blood lymphocytes (PBL) are prepared by adding PBMC to a continuous Percoll density gradient and the high density fraction collected. In some embodiments, the PBMC are concentrated and washed as described above prior to the isolation of the PBL. T cells are prepared by immediate rosetting with 2-aminoethylisothiouronium bromide-treated SRBC. T cells are further purified from rosetting cells by staining with antibodies (Abs) to CD16, CD74, and CD11 b and deleting reactive cells using immunomagnetic beads. The percentage of CD3+ cells in this fraction is usually greater than 96% (see Zheng, et al., (2002) J. Immunol., 169: 4183-4189).

In a preferred embodiment, CD8+ cells are prepared by negative selection (see Zheng, et al., (2002) J. Immunol., 169: 4183-4189).

In a preferred embodiment, CD4+ cells are prepared from T cells that are stained with Abs to CD8 by negative selection using immunomagnetic beads. The purity of CD4+ cells is usually greater that 95%. CD25-depleted T cells are prepared from CD4+ T cells by cell sorting. Before sorting, the CD4+ CD25+ population was approximately 3-5% among total CD4+ T cells. After sorting, the CD4+ CD25+ population was less than 0.3% (see Zheng, et al., (2002) J. Immunol., 169: 4183-4189).

In a preferred embodiment, the CD4+ cells are further purified to include only undifferentiated, naive CD4+ T cells. This is done by depleting the CD4+ cells of CD45RO+ cells using monoclonal antibodies.

NK T cells may be isolated by standard techniques known to those of skill in the art (see for example, Gray, et al. (1998) J. Immunol., 160: 2248, incorporated herein by reference in its entirety).

If desired, B cells can be obtained from nonrosetting PBMC treated with 5 mM _{L}-leucine methyl ester (LME) for depletion of monocytes and NK cells. The cells so obtained ar then stained with Abs to CD3, CD16, and CD11b and depleted of reactive cells by immunomagnetic beads. the resulting population is greater than 90% CD20+ and less than 0.5% CD3+ (see Zheng, et al., (2002) J. Immunol., 169: 4183-4189).

In some embodiments, T cell subsets, e.g., CD8+ or CD4+ T cells are not isolated from the PBMC until after treatment with a regulatory composition. In these embodiments, the T cells are isolated following treatment with a regulatory composition and one or more of the treated T cell subsets are returned to a patient with an immune disorder.

As will be appreciated by those of skill in the art, there are a number of other ways to isolate T cells, in addition to the preferred embodiments provided above.

Once purified or concentrated the cells may be aliquoted and frozen, preferably, in liquid nitrogen or used immediately as described below. Frozen cells may be thawed and used as needed.

Cryoprotective agents, which can be used, include but are not limited to dimethyl sulfoxide (DMSO) (Lovelock, J. E. and Bishop, M. W. H., 1959, Nature 183:1394-1395; Ashwood-Smith, M. J., 1961, Nature 190:1204-1205), hetastarch, glycerol, polyvinylpyrrolidine (Rinfret, A. P., 1960, Ann. N.Y. Acad. Sci. 85:576), polyethylene glycol (Sloviter, H. A. and Ravdin, R. G., 1962, Nature 196:548), albumin, dextran, sucrose, ethylene glycol, i-erythritol, D-ribitol, D-mannitol (Rowe, A. W., et al., 1962, Fed. Proc. 21:157), D-sorbitol, i-inositol, D-lactose, choline chloride (Bender, M. A., et al., 1960, J. Appl. Physiol. 15:520), amino acids (Phan The Tran and Bender, M. A., 1960, Exp. Cell Res. 20:651), methanol, acetamide, glycerol monoacetate (Lovelock, J. E., 1954, Biochem. J. 56:265), and inorganic salts (Phan The Tran and Bender, M. A., 1960, Proc. Soc. Exp. Biol. Med. 104:388; Phan The Tran and Bender, M. A., 1961, in Radiobiology Proceedings of the Third Australian Conference on Radiobiology, Ilbery, P. L. T., ed., Butterworth, London, p. 59). Typically, the cells may be stored in 10% DMSO, 50% serum, and 40% RPMI 1640 medium. Methods of cell separation and purification are found in U.S. Pat. No. 5,888,499, which is expressly incorporated by reference.

Generation of Regulatory T Cells

Once isolated, the T cells may be treated with a regulatory composition to generate activated regulatory T cells with enhanced suppressive activity. By “regulatory T cells” herein is meant CD8+ or CD4+ T cell subsets that develop the ability to prevent cytotoxic T cell activity in other T cells, inhibit antibody production, suppress delayed type hypersensitivity responses, inhibit monocyte, dendritic cell or B cell function as antigen presenting cells, etc. As discussed above, several regulatory T cell subsets exist. These T cell subsets can be broadly divided into two categories: (1) professional regulatory T cells; and (2) cytokine-producing regulatory cells.

By “professional regulatory T cells” herein is meant a subset of CD4+ T cells that constitutively express the alpha chain of the IL-2 receptor complex, CD25. Thus, professional regulatory T cells are CD4+ CD25+. These cells exhibit a broad range of suppressive activities including suppressing activation of other T cells, down regulating antibody production, and inhibiting cytotoxic T cell activity. These suppressive activities require the professional regulatory T cell to directly bind to other cells (i.e., contact-dependent) and deliver one or more inhibitory signals. Cytokines, such as interleukin 10 or TGF-β s are not required for the induction of these suppressive activities (i.e, cytokine-independent); thus the activity is not abolished by the addition of neutralizing monoclonal antibodies to these cytokines.

Professional regulatory T cells can be further classified as virgin or activated professional regulatory cells. By “virgin professional regulatory T cells” herein is meant professional regulatory T cells that have not been treated with the regulatory compositions described herein. By “activated professional regulatory T cells” herein is meant professional regulatory T cells that have been treated with the regulatory compositions described herein, and as a result of the treatment exhibit enhanced suppressive activity. Activated professional regulatory cells are derived from CD4+ cells.

By “cytokine producing regulatory T cells” herein is meant NK T cells, CD4+ or CD8+ Th3 or Th3-like cells that produce immunosuppressive levels of TGF-β. As these cells do not constitutively express the alpha chain of the IL-2 receptor complex, they are CD4+ CD25− cells. While cytokine producing regulatory T cells can also suppress autoimmunity, they function principally as general feedback regulators of Th1 and Th2 cells. Activated cytokine producing regulatory T cells can be generated from CD4+ cells, CD8+ cells, or NK T cells that have been treated with a regulatory composition.

In a preferred embodiment, treatment of PBMC or isolated T cell subsets increases both the number of regulatory T cells and their suppressive activity. As illustrated in FIG. 1, the number of virgin professional regulatory in a T cell population is less than 1% of the total. Following treatment with a regulatory composition, the percent of professional regulatory T cells in the population is increased from less than 1% to greater than 10%. Preferably, the percent of professional regulatory T cells in the population is increased to greater than 25%. More preferably, the percent of professional regulatory T cells in the population is increased to greater than 50%. More preferably, the percent of professional regulatory T cells in the population is increased to greater than 70%.

“Suppressive activity” herein refers to ability of a regulatory T cells to inhibit the activation of other lymphocytes, including T cells and B cells, monocytes, and dendritic cells. By “enhanced” suppressive activity” herein is meant regulatory T cells that have been activated in the presence of a regulatory composition comprising TGF-β. These regulatory T cells are able to inhibit the activity of other immune cells in fewer numbers than non-conditioned regulatory cells. The suppressive activity of professional regulatory T cells can be determined using an allogeneic mixed lymphocyte reaction as is known in the art and described in several of the Figures. For example, professional regulatory CD4+ CD25+ T cells isolated from lymphoid tissues have suppressive activity when added to other cells at 1:1 to 1:4 ratios (Shevach, E. M. (2000) Regulatory T cells in autoimmmunity. Annu. Rev. Immunol., 18, 423-449). By contrast, 1:10 to less than 1:100 activated CD4+ CD25+ cells generated in the presence of a regulatory composition comprising TGF-β exert strong suppressive effects (Yamagiwa et al, (2001) J. Immuno. 166: 7282-89).

Once isolated, the cells are treated with a regulatory composition. By “treated” herein is meant that the cells are incubated with the regulatory composition for a time period sufficient to develop regulatory T cell activity. The incubation will generally be under physiological temperature.

By “regulatory composition” herein is meant a composition that can induce T cells to suppress undesirable immune responses. By “undesirable immune responses” herein is meant immune disorders characterized by the failure of the immune system to distinguish self from non-self or to respond to foreign antigens, or immune responses to transplanted tissues. Thus, undesirable immune responses include, but are not limited to, inhibition of T cell activation, inhibition of spontaneous antibody and autoantibody production, or cytotoxicity, or both.

Regulatory compositions may comprise a number of components, including: (1) cytokines; (2) stimulator cells (i.e., irradiated T cell-depleted mononuclear cells (see U.S. Ser. No. 09/833,526); (3) T cell activators; (4) non T accessory cells; and (5) anti-cytokine neutralizing monoclonal antibodies.

The concentration of the regulatory composition will vary depending on the identity of the compounds included in the composition, but will generally be at physiologic concentration, i.e. the concentration required to give the desired effect, i.e. an enhancement of specific types of regulatory cells.

Generally, regulatory compositions include cytokines. Suitable cytokines include, but are not limited to, IL-2, IL-4, IL-5, IL-15, TGF-β and TNF-α. Preferred cytokines include IL-2, IL-15 and TGF-β.

In a preferred embodiment, TFG-β is a component the regulatory composition. By “transforming growth factor-β” or “TGF-β” herein is meant any one of the family of the TGF-βs, including the three isoforms TGF-β1, TGF-β2, and TGF-β3; see Massague, J. (1980), J. Ann. Rev. Cell Biol 6:597. Lymphocytes and monocytes produce the β1 isoform of this cytokine (Kehrl, J. H. et al. (1991), Int J Cell Cloning 9: 438-450). The TFG-β can be any form of TFG-β that is active on the mammalian cells being treated. In humans, recombinant TFG-β is currently preferred. In general, the concentration of TGF-β used ranges from about 2 picograms/ml of cell suspension to about 10 nanograms, with from about 10 pg to about 4 ng being preferred, and from about 100 pg to about 2 ng being especially preferred, and 1 ng/ml being ideal.

In a preferred embodiment, IL-2 is used in the regulatory composition. The IL-2 can be any form of IL-2 that is active on the mammalian cells being treated. In humans, recombinant IL-2 is currently preferred. In general, the concentration of IL-2 used ranges from about 1 Unit/ml of cell suspension to about 100 U/ml, with from about 5 U/ml to about 25 U/ml being preferred, and with 10 U/ml being especially preferred. In a preferred embodiment, IL-2 is not used alone.

In a preferred embodiment, IL-15 is used in the regulatory composition. The IL-15 can be any form of IL-15 that is active on the mammalian cells being treated. In humans, recombinant IL-15 is currently preferred. In general, the concentration of IL-15 used ranges from about 1 Unit/ml of cell suspension to about 100 U/ml, with from about 5 U/ml to about 25 U/ml being preferred, and with 10 U/ml being especially preferred. In a preferred embodiment, IL-15 is not used alone.

In a preferred embodiment, the regulatory composition comprises T cell activators. Suitable T cell activators include soluble antigens, peptide fragments of antigens, alloantigens, anti-CD2, anti-CD3, anti-CD28, LFA-3, and mitogens. As will be appreciated by those of skill in the art, anti-CD3, soluble antigens, and peptide fragments of antigens are T cell receptor (TCR) activators.

CD2 is a cell surface glycoprotein expressed by T lymphocytes. By “CD2 activator” herein is meant compound that will initiate the CD2 signaling pathway. A preferred CD2 activator comprises anti-CD2 antibodies (OKT11, American Type Culture Collection, Rockville Md. and GT2, Huets, et al., (1986) J. Immunol. 137:1420). In addition, a combination of anti-CD2 antibodies can be used, including the CD2 ligand LFA-3, in the regulatory composition. In general, the concentration of CD2 activator used will be sufficient to induce the production of TGF-β. The concentration of anti-CD2 antibodies used ranges from about 1 ng/ml to about 10 μg/ml, with from about 10 ng/ml to about 100 ng/ml being especially preferred.

In some embodiments it is desirable to use a mitogen to activate the cells; that is, many resting phase cells do not contain large amounts of cytokine receptors. The use of a mitogen such as Concanavalin A (ConA) or staphylococcus enterotoxin B (SEB) can allow the stimulation of the cells to produce cytokine receptors, which in turn makes the methods of the invention more effective. When a mitogen is used, it is generally used as is known in the art, at concentrations ranging from 1 pg/ml to about 10 pg/ml is used. In addition, it may be desirable to wash the cells with components to remove the mitogen, such as α-methyl mannoside, as is known in the art.

In a preferred embodiment, non T accessory cells or an equivalent surrogate are used in the regulatory composition. Non T accessory cells that may be included in the regulatory composition are B cells, macrophages, monocytes, and dendritic cells.

In a preferred embodiment, anti-cytokine neutralizing monoclonal antibodies are used in the regulatory composition. Suitable anti-cytokine neutralizing monoclonal antibodies include anti-TGF-β.

In some embodiments, such as the generation of regulatory T cells for use in graft rejection or GVHD, stimulator cells (e.g. irradiated T cell depleted cells) are included in the regulatory composition (see U.S. Ser. Nos. 09/653,924 and 09/833,526).

Accordingly, a regulatory composition comprising at least one of the above components may be used to generate activated regulatory T cells. The regulatory compositions may contain more than one compound from the same class of compounds, i.e., two or more cytokines, may be mixed together. The composition also may contain compounds from different classes of compounds, such as a cytokine and a T cell activator, etc. Thus, regulatory compositions containing: (1) one cytokine; (2) two or more cytokines; (3) at least one cytokine, and a T cell activator; (4) at least one cytokine, a T cell activator, and a stimulator; (5) at least one cytokine, a T cell activator, and non T accessory cells; and (6) at least one cytokine, a T cell activator, and an anti-cytokine antibody, (7) at least one cytokine and at least one T-cell activator with or without non T accessory cells; and (8) at least one cytokine, at least one T-cell activator, at least one anti-cytokine antibody, with or without non T accessory cells; and (9) at least one cytokine, at least one T-cell activator, at least one stimulator, at least one anti-cytokine antibody, with or without non T accessory cells, may be used to generate activated regulatory T cells. As will be appreciated by those of skill in the art, the above list of combinations is not meant to be exhaustive, but is provided as examples for the possible combinations of components that may be included in the regulatory compositions of the present invention.

As will be appreciated by those of skill in the art, the combination that is used will depend on whether T cell proliferation, T cell differentiation, or both is the desired outcome. Moreover, the number of cytokine-producing regulatory cells to professional regulatory cells that are generated by a given treatment will vary depending on the percentage of CD4+ CD25+ cells in the starting population, the nature of the T cell activator and signals provided by non-T accessory cells.

In a preferred embodiment, IL-2 and TGF-β are used together to generate activated regulatory T cells with enhanced suppressive activity. As will be appreciated by those of skill in the art, both professional regulatory T cells and cytokine-producing T cells are produced using this regulatory composition. Moreover, professional regulatory T cells and cytokine-producing T cells may be generated from various T cell subsets, including CD4+, CD8+, naïve CD4+ cells, etc.

In a preferred embodiment, IL-15 and TGF-β are used together to generate activated regulatory T cells with enhanced suppressive activity. As will be appreciated by those of skill in the art, both professional regulatory T cells and cytokine-producing T cells are produced using this regulatory composition. Moreover, professional regulatory T cells and cytokine-producing T cells may be generated from various T cell subsets, including CD4+, CD8+, naïve CD4+ cells, etc.

In a preferred embodiment, IL-2, TGF-β, and a CD2 activator, are used to generate activated regulatory T cells with enhanced suppressive activity. As will be appreciated by those of skill in the art, both professional regulatory T cells and cytokine-producing T cells are produced using this regulatory composition. Moreover, professional regulatory T cells and cytokine-producing T cells may be generated from various T cell subsets, including CD4+, CD8+, naïve CD4+ cells, etc. Other preferred combinations include IL-15, TGF-β, and a CD2 activator.

In a preferred embodiment, IL-2, IL-15, and TGF-β are used to generate activated regulatory T cells with enhanced suppressive activity.

In a preferred embodiment, IL-2, TGF-β, and an anti-cytokine antibody are used to generate activated regulatory T cells with enhanced suppressive activity. Other preferred combinations include IL-15, TGF-β, and an anti-cytokine.

In a preferred embodiment, IL-2, TGF-β, and a TCR activator are used to generate T cells with suppressive activity. Other preferred combinations include IL-2, TGF-β, a TCR activator, and non T accessory cells; IL-15, TGF-β, and a TCR activator; IL-15, TGF-β, a TCR activator, and non T accessory cells.

As will be appreciated by those of skill in the art, repeated stimulation of the T cells with our without a regulatory composition in secondary cultures may be necessary to develop maximal suppressive activity.

In a preferred embodiment, T cell activators are used to generate cytokine producing regulatory T cells. In this embodiment, the T cell activators are used in combination with conventional T cells and activated professional regulatory T cells to generate activated cytokine producing regulatory T cells.

Once the cells have been treated, they may be evaluated for suppressive activity and suitability for transplantation into a patient. For example, a sample may be removed for: sterility testing; gram staining, microbiological studies; LAL studies; mycoplasma studies; flow cytometry to identify cell types; functional studies, etc. These and other lymphocyte studies may be done before and after treatment. A preferred analysis is to label a test or target population of cells that are capable of eliciting an immune response in the treated T cells, incubate the treated T cells with the labeled population, and determine cell survival as a measure of suppressive activity (see Figures). Assays, such as those described in Example 1 and in the brief description of the Figures may be used to determine suppressive activity.

Assays such as those described in U.S. Pat. No. 6,358,506, incorporated herein by reference in its entirety, can also be used to identify professional regulatory T cells. The addition of neutralizing antibodies and IL-10 to a population of treated cells can be used to identify cytokine-producing T cells (see Example 1).

Uses for Regulatory T Cells

Once generated, T cells with suppressive activity may be administered to alleviate an immune response in a patient. By “immune response” herein is meant host responses to foreign or self antigens. Preferably, T cells with suppressive activity are used to prevent aberrant immune response or undesirable immune responses to foreign antigens. By “aberrant immune responses” herein is meant the failure of the immune system to distinguish self from non-self or the failure to respond to foreign antigens. In other words, aberrant immune responses are inappropriately regulated immune responses that lead to patient symptoms. By “inappropriately regulated” herein is meant inappropriately induced, inappropriately suppressed and/or non-responsiveness. Aberrant immune responses include, but are not limited to, tissue injury and inflammation caused by the production of antibodies to an organism's own tissue, impaired production of IL-2, TNF-α and IFN-γ and tissue damage caused by cytotoxic or non-cytotoxic mechanisms of action. By “undesirable immune responses” herein is the responses to foreign antigens observed in transplant patients. Thus, undesirable immune responses include responses associated with GVHD and graft rejection.

By “patient” herein is meant a mammalian subject to be treated, with human patients being preferred. In some cases, the methods of the invention find use in experimental animals, in veterinary applications, and in the development of animal models for disease, including, but not limited to, rodents including mice, rats, and hamsters; and primates.

In a preferred embodiment, the present invention inhibits aberrant immune responses. In patients with antibody-mediated autoimmune disorders, the present invention restores the capacity of peripheral blood T cells to down regulate antibody production and restores cell mediated immune responses by treating them with an regulatory composition ex vivo. In patients with cell-mediated disorders, the present invention generates regulatory T cells which suppress cytotoxic T cell activity in other T cells.

Accordingly, in a preferred embodiment, the present invention provides methods of treating antibody-mediated autoimmune disorders in a patient. By “antibody-mediated autoimmune diseases” herein is meant a disease in which individuals develop antibodies to constituents of their own cells or tissues. Antibody-mediated autoimmune diseases include, but are not limited to, systemic lupus erythematosus (SLE), pemphigus vulgaris, myasthenia gravis, hemolytic anemia, thrombocytopenia purpura, Grave's disease, dermatomyositis and Sjogren's disease. The preferred autoimmune disease for treatment using the methods of the invention is SLE.

In addition, patients with antibody-mediated disorders frequently have defects in cell-mediated immune responses. By “defects in cell mediated immune response” herein is meant impaired host defense against infection. Impaired host defense against infection includes, but is not limited to, impaired delayed hypersensitivity, impaired T cell cytotoxicity and impaired production of TGF-β. Other defects, include, but are not limited to, increased production of IL-10 and decreased production of IL-2, TNF-α and IFN-γ. Using the methods of the present invention, purified T cells are stimulated to increase production of IL-2, TNF-α and IFN-γ and decrease production of IL-10. T cells which can be stimulated using the current methods include, but are not limited to, CD4+ and CD8+.

In one embodiment, antibody-mediated disorders are not treated.

In a preferred embodiment, the present invention provides methods of treating cell-mediated autoimmune disorders in a patient. By “cell-mediated autoimmune diseases” herein is meant a disease in which the cells of an individual are activated or stimulated to become cytotoxic and attack their own cells or tissues. Alternatively, the autoimmune cells of the individual may stimulate other cells to cause tissue damage by cytotoxic or non-cytotoxic mechanisms of action. Cell-mediated autoimmune diseases include, but are not limited to, Hashimoto's disease, polymyositis, disease inflammatory bowel disease, multiple sclerosis, diabetes mellitus, rheumatoid arthritis, and scleroderma.

By “treating” an autoimmune disorder herein is meant that at least one symptom of the autoimmune disorder is ameliorated by the methods outlined herein. This may be evaluated in a number of ways, including both objective and subjective factors on the part of the patient. For example, immunological manifestations of disease can be evaluated; for example, the level of spontaneous antibody and autoantibody production, particularly IgG production in the case of SLE, is reduced. Total antibody levels may be measured, or autoantibodies, including, but not limited to, anti-double-stranded DNA (ds DNA) antibodies, anti-nucleoprotein antibodies, anti-Sm, anti-Rho, and anti-La. Cytotoxic activity can be evaluated as outlined herein. Physical symptoms may be altered, such as the disappearance or reduction in a rash in SLE. Renal function tests may be performed to determine alterations; laboratory evidence of tissue damage relating to inflammation may be evaluated. Decreased levels of circulating immune complexes and levels of serum complement are further evidence of improvement. In the case of SLE, a lessening of anemia may be seen. The ability to decrease a patient's otherwise required drugs such as immunosuppressives can also be an indication of successful treatment. Other evaluations of successful treatment will be apparent to those of skill in the art of the particular autoimmune disease.

In a preferred embodiment, the quantity or quality, i.e. type, of antibody production, may be evaluated. Thus, for example, total levels of antibody may be evaluated, or levels of specific types of antibodies, for example, IgA, IgG, IgM, anti-DNA autoantibodies, anti-nucleoprotein (NP) antibodies, etc. may be evaluated. Regulatory T cells may also be assessed for their ability to suppress T cell activation or to prevent T cell cytotoxicity against specific target cells in vitro (see U.S. Pat. No. 6,358,506, incorporated herein by reference in its entirety).

After the treatment, the cells are transplanted or reintroduced back into the patient. This is generally done as is known in the art, and usually comprises injecting or introducing the treated cells back into the patient, via intravenous administration, as will be appreciated by those in the art. For example, the cells may be placed in a 50 ml Fenwall infusion bag by injection using sterile syringes or other sterile transfer mechanisms. The cells can then be immediately infused via IV administration over a period of time, such as 15 minutes, into a free flow IV line into the patient. In some embodiments, additional reagents such as buffers or salts may be added as well.

After reintroducing the cells into the patient, the effect of the treatment may be evaluated, if desired, as is generally outlined above. Thus, evaluating immunological manifestations of the disease may be done; for example the titers of total antibody or of specific immunoglobulins, renal function tests, tissue damage evaluation, etc. may be done. Tests of T cells function such as T cell numbers, phenotype, activation state and ability to respond to antigens and/or mitogens also may be done.

The treatment may be repeated as needed or required. For example, the treatment may be done once a week for a period of weeks, or multiple times a week for a period of time, for example 3-5 times over a two week period. Generally, the amelioration of the autoimmune disease symptoms persists for some period of time, preferably at least months. Over time, the patient may experience a relapse of symptoms, at which point the treatments may be repeated.

Kits

In a preferred embodiment, the invention further provides kits for the practice of the methods of the invention, i.e., the incubation of cells with the regulatory compositions. The kit may have a number of components. For example, the kit may comprise a cell treatment container that is adapted to receive cells from a patient with an antibody-mediated or cell-mediated autoimmune disorder. The container should be sterile. In some embodiments, the cell treatment container is used for collection of the cells, for example it is adaptable to be hooked up to a leukophoresis machine using an inlet port. In other embodiments, a separate cell collection container may be used.

In a preferred embodiment, the kit may also be adapted for use in a automated closed system to purify specific T cell subsets and expand them for transfer back to the patient.

The form and composition of the cell treatment container may vary, as will be appreciated by those in the art. Generally the container may be in a number of different forms, including a flexible bag, similar to an IV bag, or a rigid container similar to a cell culture vessel. It may be configured to allow stirring. Generally, the composition of the container will be any suitable, biologically inert material, such as glass or plastic, including polypropylene, polyethylene, etc. The cell treatment container may have one or more inlet or outlet ports, for the introduction or removal of cells, reagents, regulatory compositions, etc. For example, the container may comprise a sampling port for the removal of a fraction of the cells for analysis prior to reintroduction into the patient. Similarly, the container may comprise an exit port to allow introduction of the cells into the patient; for example, the container may comprise an adapter for attachment to an IV setup.

The kit further comprises at least one dose of an regulatory composition. “Dose” in this context means an amount of the regulatory composition such as cytokines, that is sufficient to cause an effect. In some cases, multiple doses may be included. In one embodiment, the dose may be added to the cell treatment container using a port; alternatively, in a preferred embodiment, the dose is already present in the cell treatment container. In a preferred embodiment, the dose is in a lyophilized form for stability, that can be reconstituted using the cell media, or other reagents.

In some embodiments, the kit may additionally comprise at least one reagent, including buffers, salts, media, proteins, drugs, etc. For example, mitogens, monoclonal antibodies and treated magnetic beads for cell separation can be included.

In some embodiments, the kit comprise written instructions for using the kit.

The following examples serve to more fully describe the manner of using the above described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### EXAMPLES

### Example 1

### Effects of TGF-β Co-Stimulation on CD4+ and CD8+ T Cells

Effect on Growth of CD4+ and CD8+ Cells

As shown in FIG. 1, co-stimulation by TGF-β markedly increases the percentage and absolute numbers of total CD4+ CD25+ and CD4+ CD25− cells. However, the increase in CD4+ CD25− cells was dependent upon the CD4+ CD25+ subset, as depletion of the CD4+ CD25+ subset abolished the growth promoting effects of TGF-β. A similar, but smaller effect was observed with CD8+ cells.

In these experiments, CD4+ or CD8+ cells were depleted of CD25+ cells by staining with anti-CD25. Stained cells were removed using immunomagnetic beads. Total T cell subsets and CD25 depleted T cell subsets were mixed with allogeneic irradiated non-T cells and cultured for 6 days with graded amounts of TGF-β. At the conclusion of the culture period, the total number of each subset and those that expressed CD25 was determined.

Effect on the Expansion of CD4+ Expressing Different Cell Surface Markers

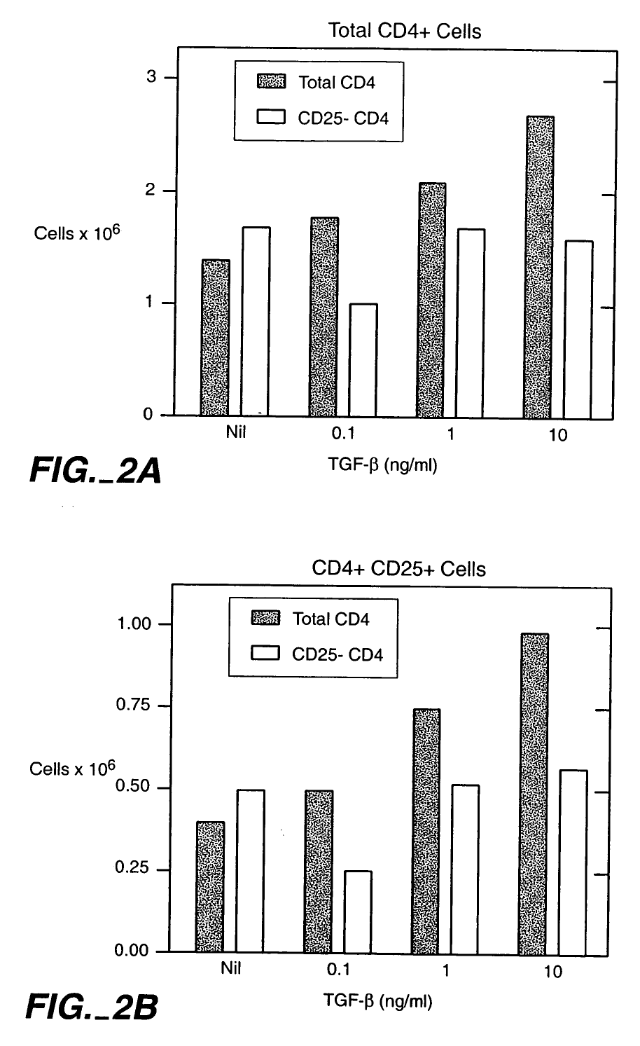

FIGS. 2A and 2B illustrate the expression of cell surface markers on CD4+ subsets after stimulation by TGF-β in an allogeneic mixed lymphocyte reaction. Total CD4+ cells, CD4+ cells depleted of CD25, and naïve CD45RA+ CD45RO− cells were studied. A dose dependent effect of TGF-β on the expression CD25 on total CD4+ cells and naïve cells was observed. Depletion of CD25 in the starting population abolished this effect. Thus, TGF-β, appears to expand the CD4+ CD25+ subset of CD4+ cells.

A similar TGF-β dose-dependent effect was observed in the expression of CD62L (L selectin) on CD4+ subsets. This result is consistent with the results of others showing that CD62L is expressed by professional CD$+ CD25+ cells. Co-stimulatory effects of TGF-β were also in CD4+ CD25− cells.

In addition, TGF-β also increased the expression of CTLA-4 and CD122, the β chain of the IL-2 receptor.

Effect on Suppressive Activity of CD4+ Subsets

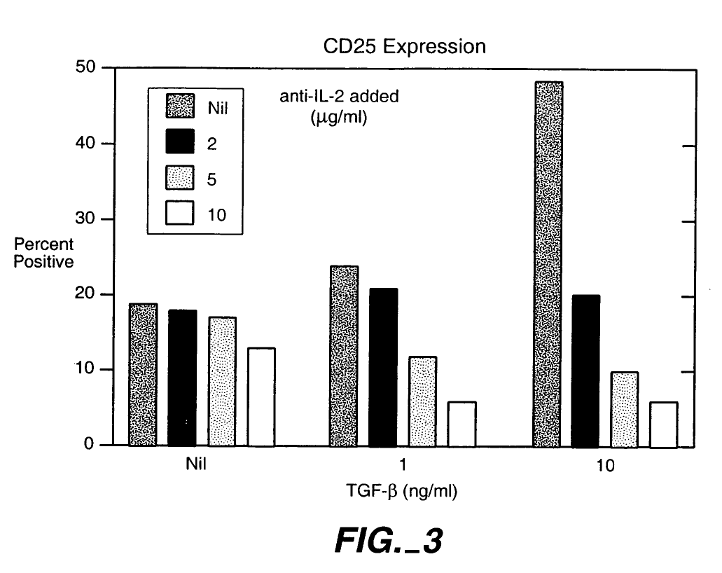

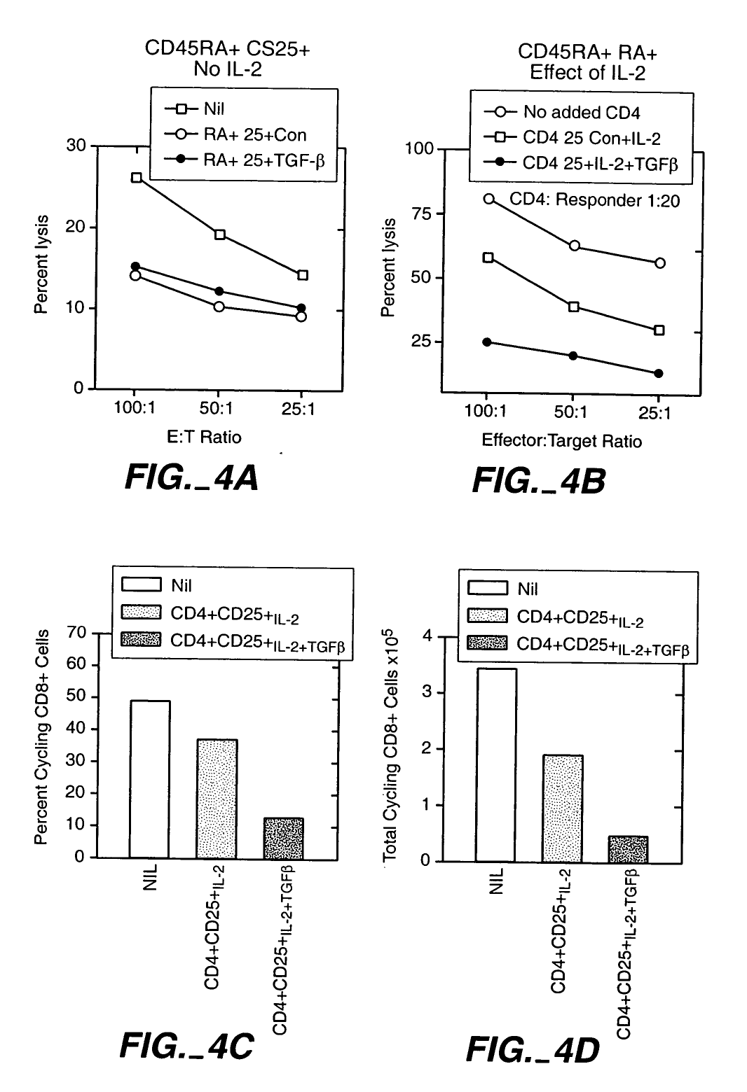

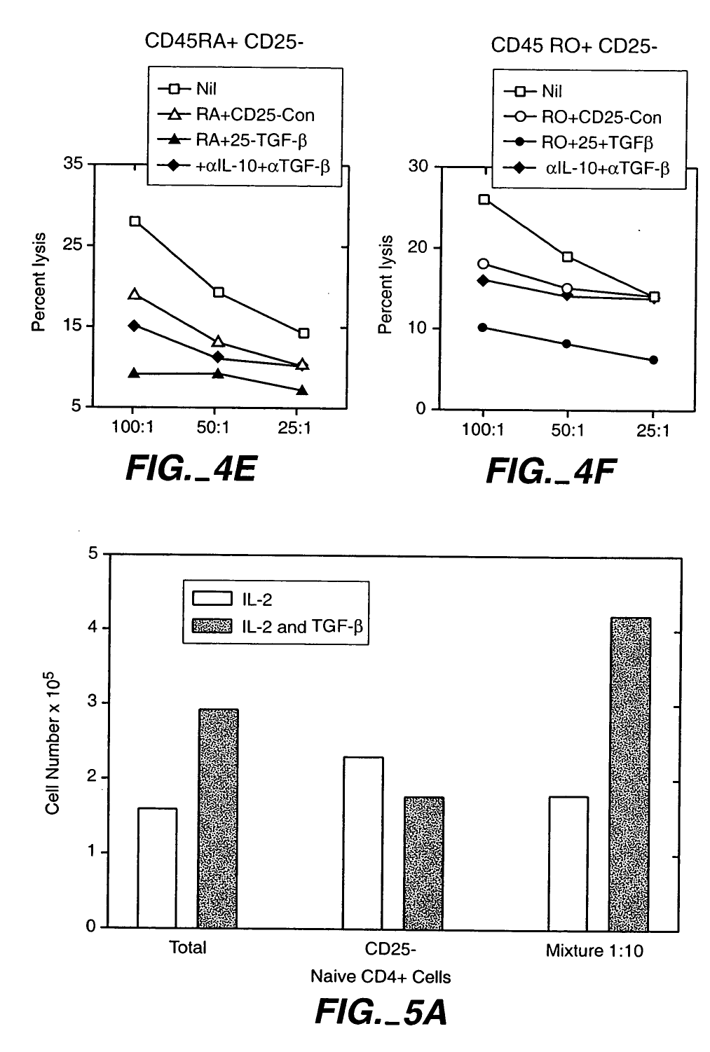

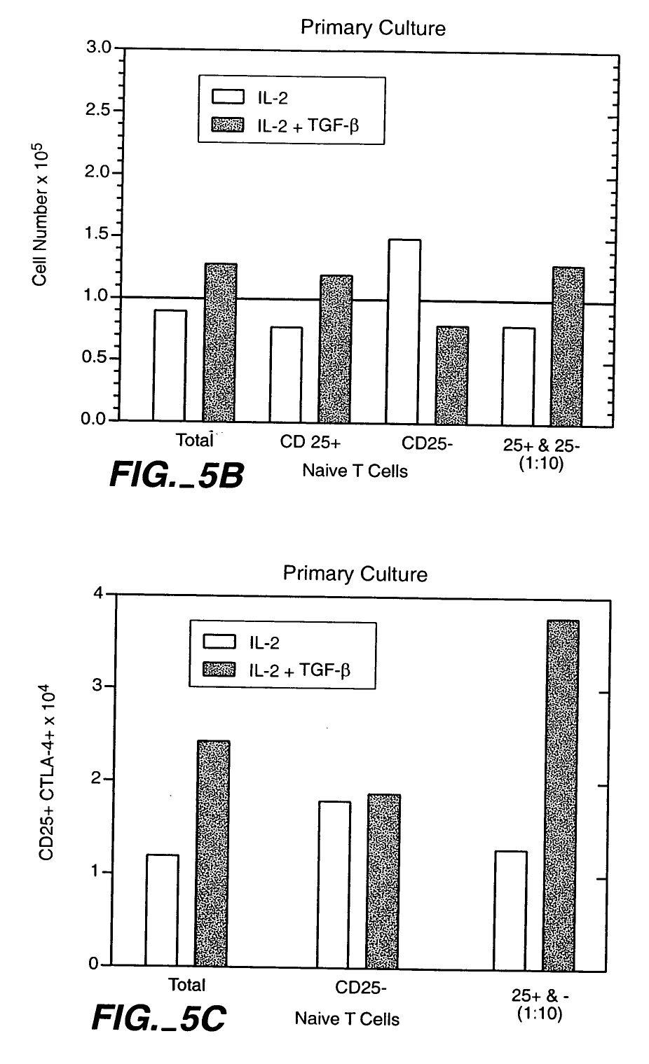

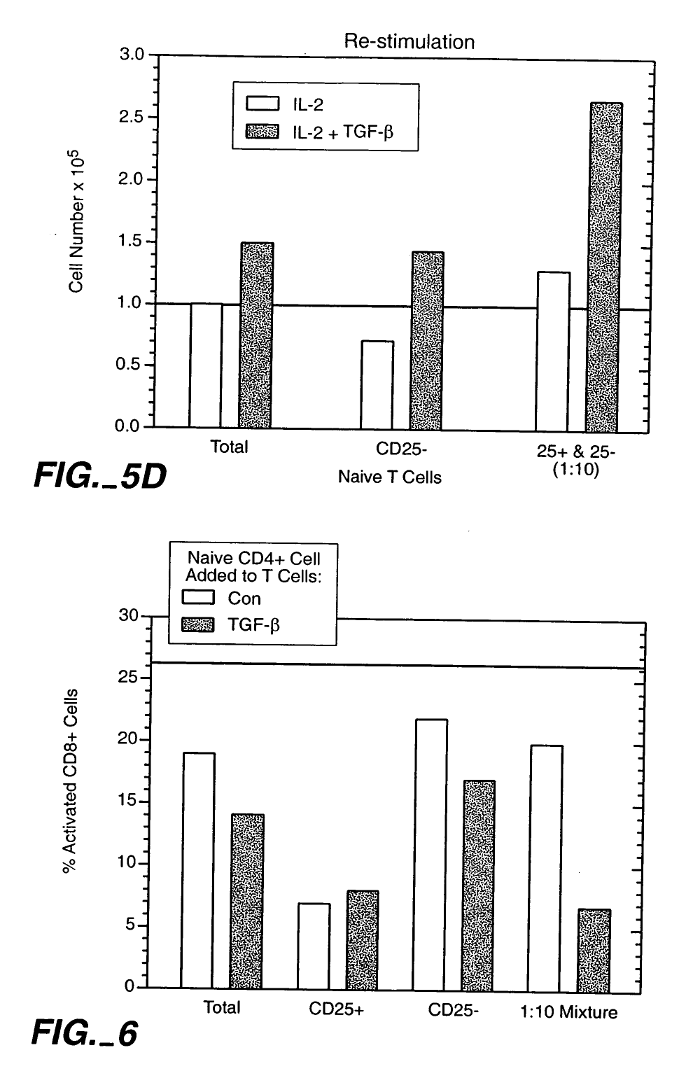

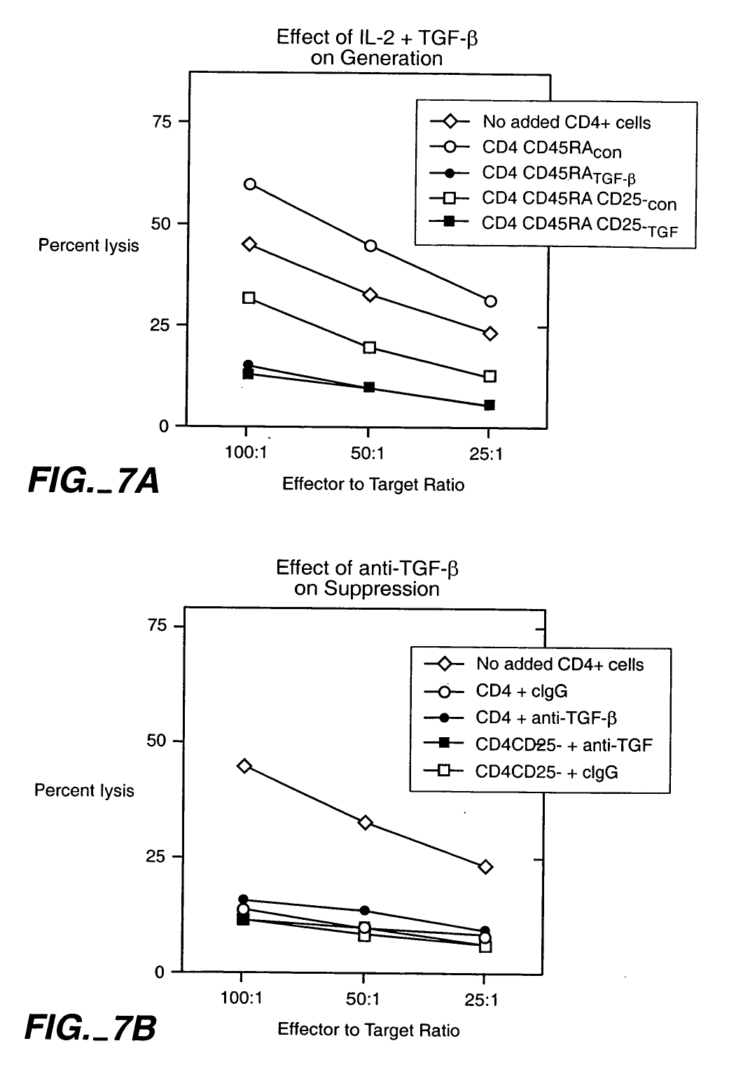

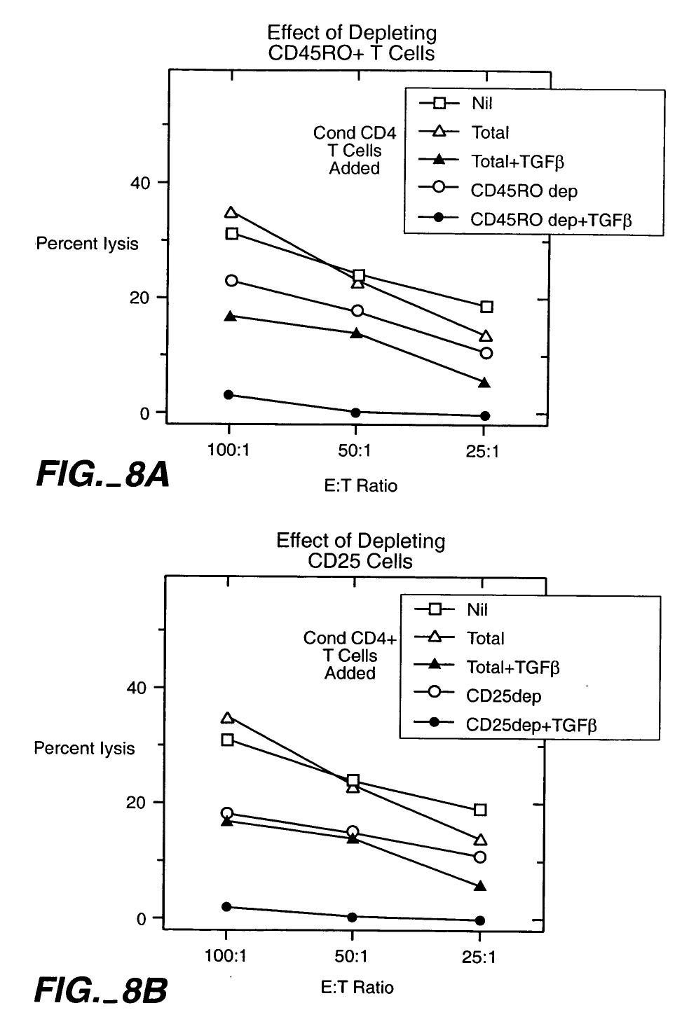

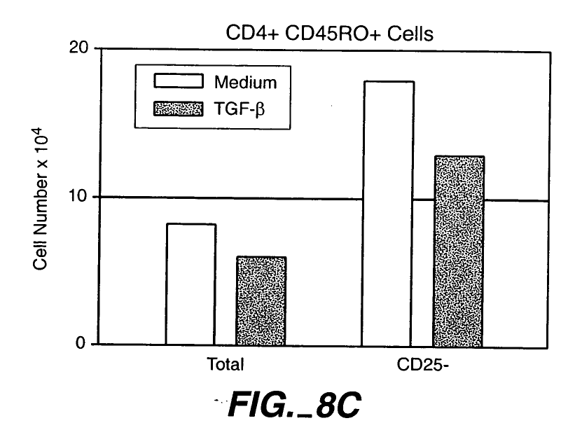

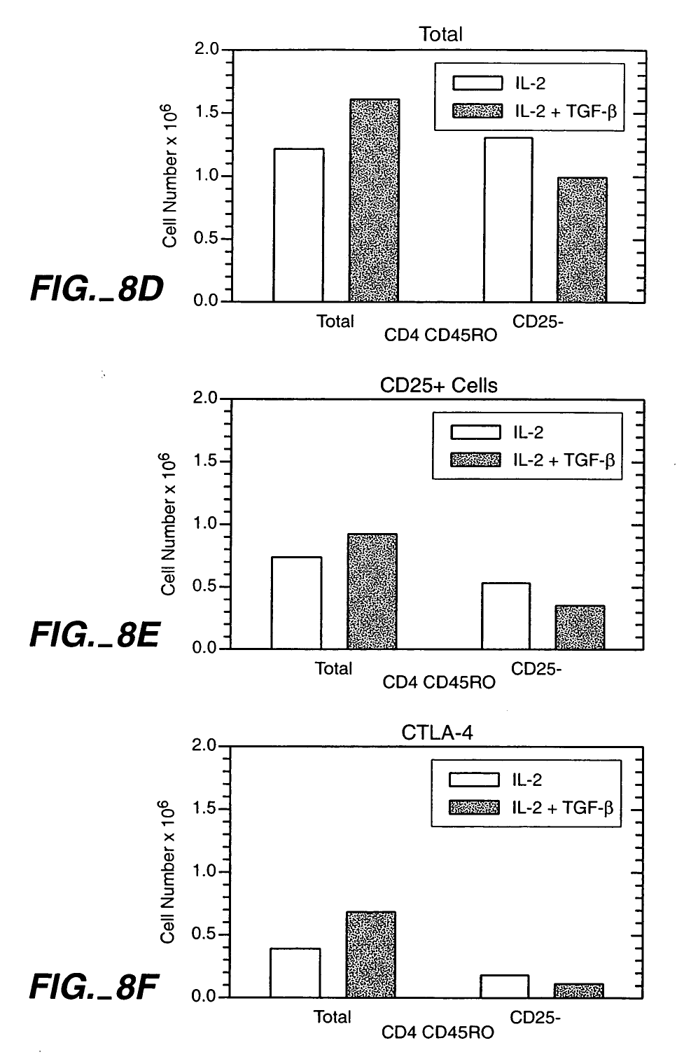

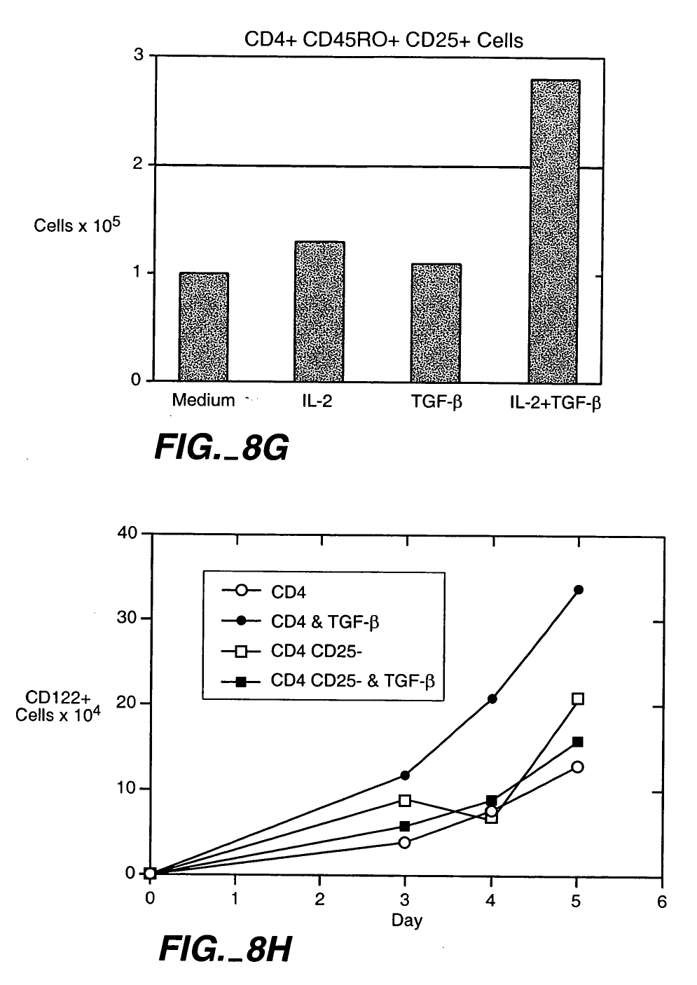

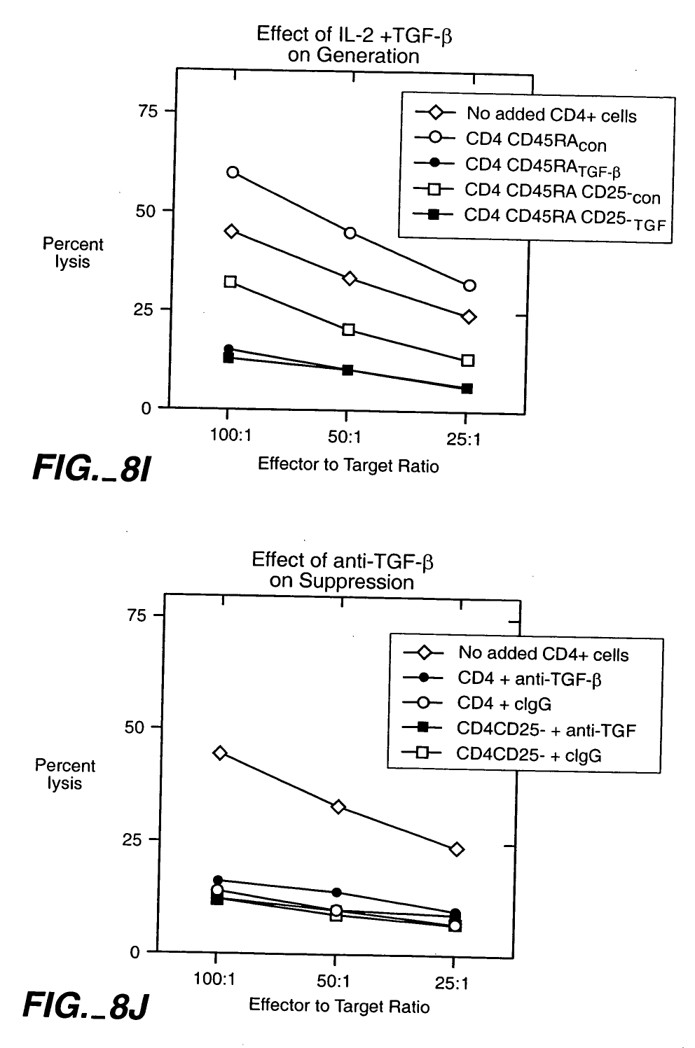

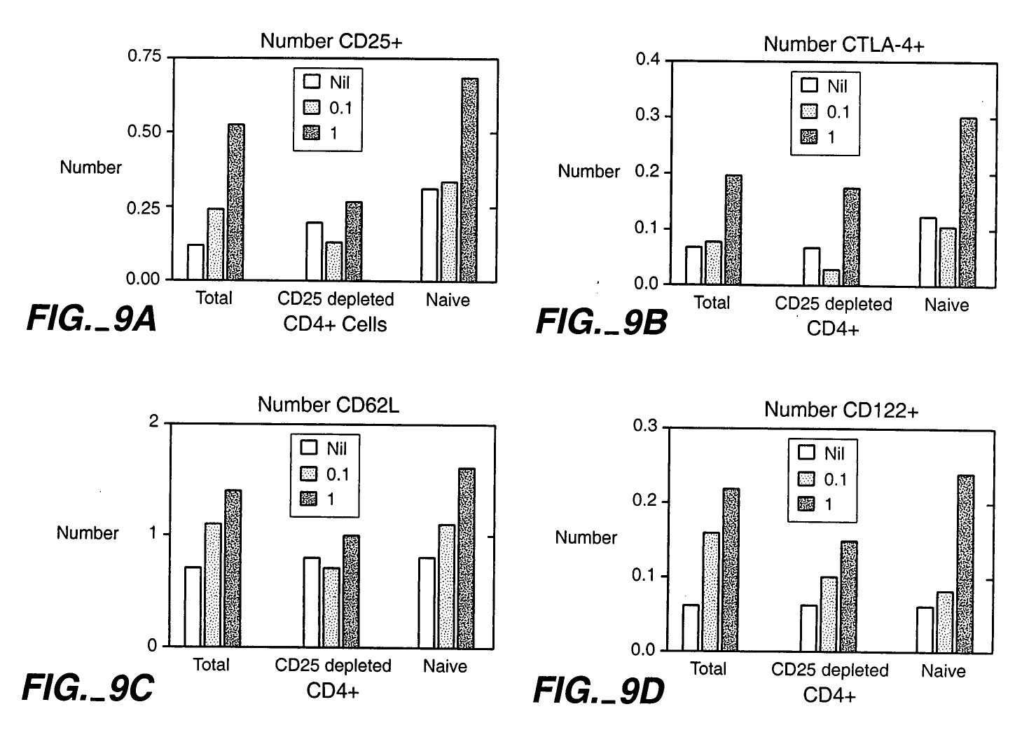

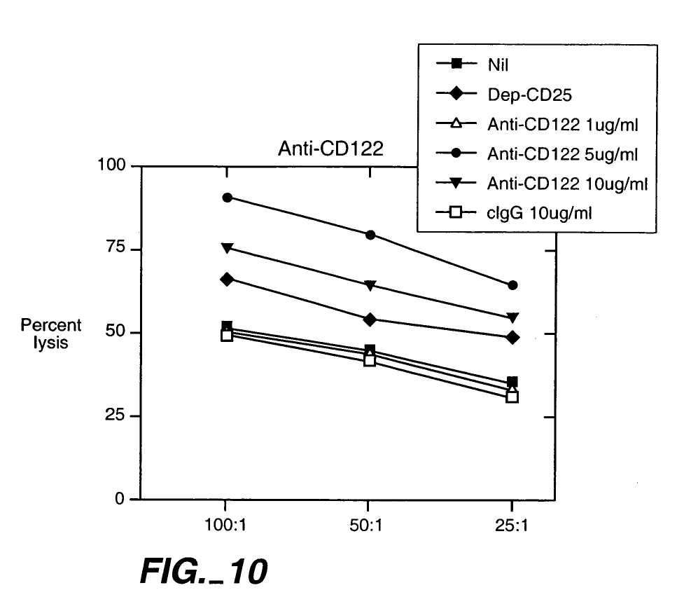

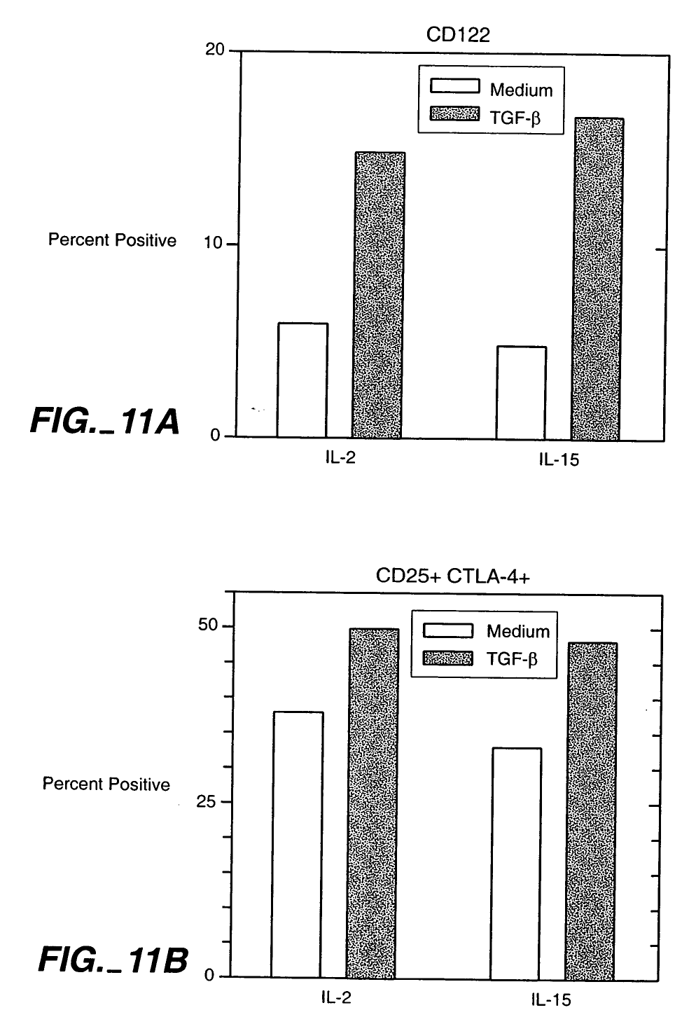

FIGS. 3A-D depict the effect of TGF-β in inducing suppressive activity by various CD4+ T cell subsets. In these experiments, purified CD4+ T cell subsets were obtained by cell sorting and conditioned with TGF-β (1 ng/ml) in an allo mixed lymphocyte reaction (MLR) as described above. The purified CD4+ subsets were thene tested for their ability to inhibit the generation of T cell cytotoxicity. FIGS. 3A and B show that purified CD4+ CD25+ T cells have significant suppressive acitivty and that this activity is significantly increased, i.e., enhanced, by conditioning with TGF-β. FIGS. 3C and 3D show the TGF-β has similar effects, i.e., a marked increase in the suppressive activity, on other T cell subsets: CD45RA+ CD45RO− CD25−, and CD45RA− CD45RO+ subsets of CD4+ cells. Addition of IL-2 is not required for this enhancement of suppressive activity.

The addition of neutralizing monoclonal antibodies and IL-10 blocked the suppressive activity of these cells, suggesting that al least some of the observed suppressive activity is cytokine-dependent.

What is claimed is:

1. A method for treating an undesirable or aberrant immune response in an individual comprising
a) treating a population of CD4+ T cells comprising conventional CD25− T cells and professional CD25+ T cells with a regulatory composition comprising TGF-beta for a period sufficient to increase the number and/or suppressive activity of the regulatory/suppressor T cells obtained by said treating; and
b) administering said regulatory/suppressor T cells to an individual exhibiting an undesirable or aberrant immune response.

2. The method of claim 1 wherein said regulatory composition further comprises and at least one other cytokine

3. The method of claim 2 wherein said cytokine is IL-2 or IL-15.

4. The method of claim 2 wherein said at least one other cytokine comprises IL-2.

5. The method of claim 2 further comprising at least one T cell activator.

6. The method of claim 5 wherein said T cell activator is selected from the group consisting of soluble antigens, peptide fragments of antigens, alloantigens, anti-CD2, anti-CD3 and staphylococcus enterotoxin B.

7. The method of claim 1 wherein said regulatory composition further comprises anti-CD28 or IFA-3.

8. The method of claim 1 wherein said regulatory composition further comprises anti-CD28.

9. The method of claim 1 wherein said treating induces the expression of CD122 on the surface of said regulatory T cells.

10. The method of claim 1 wherein said regulatory composition further comprises at least one population of non T accessory cells.

11. The method of claim 10 wherein said non T accessory cells are selected from the group consisting of B cells, macrophages, monocytes and dendritic cells.

12. The method of claim 1 wherein prior to step (b) other conventional CD25− T cells are treated with the regulatory/suppressor cells of step (a) to produce additional regulatory/suppressor T cells, wherein the formation of said additional regulatory/suppressor T cells is not dependent on the use of a regulatory composition.
